(19) United States
(12) Patent Application Publication
(10) Pub. No.: US 2011/0257477 A1
(43) Pub. Date: Oct. 20, 2011

(54) Endoscopic Mucosal Resection (EMR) Over-Sheath and Methods

(75) Inventor: John McWeeney, Brighton, MA (US)

(73) Assignee: Beacon Endoscopic Corporation, Danvers, MA (US)

(21) Appl. No.: 13/090,068

(22) Filed: Apr. 19, 2011

Related U.S. Application Data
(60) Provisional application No. 61/325,406, filed on Apr. 19, 2010.

(51) Int. Cl. A61B 1/00

(52) U.S. Cl. 600/104

(57) ABSTRACT

The present invention provides methods and devices for facilitating endoscopic tissue resection procedures. The devices of the invention include a flexible over-sheath having one or more lumens/channels and/or a rigid or semi-rigid end cap element for coupling to an endoscopic device.

### REFERENCE TO RELATED APPLICATIONS

This non-provisional application claims priority under 35 U.S.C. 119(e) to Provisional Patent Application No. 61/325,406, filed Apr. 19, 2010, the contents of which are herein incorporated by reference in its entirety.

### TECHNICAL FIELD

The present disclosure generally relates to the Endoscopic Mucosal Resection (EMR), and more particularly, to over-sheath devices to aid in polyp resection & removal needle conjunction with endoscopic techniques.

### BACKGROUND OF THE INVENTION

Endoscopic mucosal resection (EMR), which was developed as a therapeutic method for resection of flat or depressed lesions of the gastrointestinal tract (esophagus), has also come to be widely accepted as an effective treatment for early colon cancer. EMR can be curative when the cancer, limited to the mucosa, is completely excised and lymph node metastasis is absent. In recent years, progress has been made in the development of EMR techniques. Various techniques and instruments have been devised to make EMR safer and more effective. If a small polyp-like nodule is seen in the esophagus, an endoscopic mucosal resection (EMR) can be performed to try to remove it. The nodule is first injected with a solution that will decrease bleeding after the nodule is removed. This solution also forms a blister under the nodule to allow the physician to remove same without damaging the rest of the esophagus. Similar techniques are also utilized in the treatment of colorectal cancer.

Typical EMR procedures are conducted using a small cap (e.g., those provided by Olympus Corp. Tokyo, Japan) which has a small wire loop which fits on the end of the endoscope. The nodule is suctioned into the cap and the wire loop is closed while electro-cautery energy is applied. This is performed so that the tissue can be examined under a microscope to determine if all cancer (or dysplasia) has been removed. In order to utilize the aforementioned cap device to resect the desired tissue, the Cap is preloaded onto the distal end of an endoscope or colonoscope prior to insertion to the desired resection site. Once the sample has been resected the specimen or polyp is removed. In the event that the polyp or specimen is too large to be removed through the working channel of the endoscope/colonoscope, the endoscope/colonoscope must be entirely removed from the patient. If it is desirable to resect a second specimen from the patient, the physician must re-insert the endoscope/colonoscope once again to the desired location. This effort can be time consuming, particularly in the case of colorectal EMR where navigation is impaired by the tortuous nature of the anatomy.

A clinical need therefore exists for an EMR device which can improve the efficiency of EMR procedures, particularly colonic EMR procedures, by providing the physician with embodiments to remove sample specimens without needing to remove the endoscope/colonoscope after each sample acquisition.

### SUMMARY OF INVENTION

The present invention provides devices and methods for facilitating EMR procedures. The devices of the invention include an over-sheath device having one or more channels/lumens and/or an end cap element for coupling to an endoscope or a working channel of a colonoscope. The devices of the invention allow resected polyps to be removed/retrieved without removing the endoscope. The devices of the invention also allow for the removal and retrieval of multiple polyps without having to remove and re-insert the endoscope/colonoscope.

In one embodiment, the invention provides an over-sheath device having at least one channel/lumen and including an end-cap element. The over-sheath device includes a flexible elongate member having proximal portion and a distal portion. A primary channel/lumen extends along the length of the elongate member, the primary channel/lumen having an inner surface and an outer surface defined by the elongate member. The proximal portion of the primary channel/lumen is configured for housing an endoscope or a channel of an endoscopic device (e.g., a working channel of a colonoscope). The distal portion includes a rigid or semi-rigid end cap element. In certain embodiments, the distal cap member is integrated with the elongate member of the over-sheath device to form a smooth transition between the elongate member and cap element. In other embodiments, the distal cap element is a separate element coupled or bonded to the distal portion of the device. The primary channel/lumen of the elongate member can have a C-shape or a U-shaped cross-section. Alternatively, the primary channel/lumen of the elongate member is an enclosed lumen.

The flexible elongate member of the over-sheath device can further include a secondary side channel/lumen disposed alongside the outer surface of the primary lumen and distally extending to the distal end of the end cap element. Preferably the secondary side channel/lumen has a diameter smaller than a diameter of the primary lumen. The secondary side channel/lumen can have a C-shape or U-shaped cross section. Alternatively, the secondary side channel/lumen of the elongate member is an enclosed lumen.

In certain embodiments, the secondary side channel/lumen includes a deflectable distal tip portion. In such embodiments, the flexible elongate member of the over-sheath device further includes a lumen disposed within a wall of the secondary side channel. This lumen extends alongside the secondary side channel in a distal direction to the distal end of the end cap element. The lumen houses a pull wire that is coupled (e.g., bonded) to the distal tip portion of the secondary side channel/lumen. The lumen/pull wire (extends in a proximal direction to an actuator configured for deflecting the distal tip of the secondary side channel/lumen (the secondary side channel/lumen also extending in a proximal direction alongside the lumen/pull wire)). In certain embodiments, the actuator is a push/pull handle that deflects the distal tip in outward, inward and neutral directions. For example, the handle can be configured to deflect the distal tip in an outward direction relative the end cap when the handle is pulled in an upward direction, and inward relative to the end cap when the handle is pushed in a distal direction. Alternatively, the push/pull handle is configured to deflect the distal tip in an inward direction relative the end cap when the handle is pulled in an upward direction, and outward relative to the end cap when the handle is pushed in a distal direction.

The invention further provides a device for endoscopic mucosal resection comprising a rigid or semi-rigid end cap. The end cap is configured for housing an endoscope or a channel of an endoscopic device (e.g., a working channel of a colonoscope) and includes a side channel that is either integrated into the end cap, or disposed within or along an outer surface of said end cap. The side channel is preferably a C-shaped or a U-shaped channel. The side channel can be used to pass ancillary devices alongside the endoscope/working channel to facilitate an endoscopic procedure.

The invention even further provides a dual-lumen over-sheath device without an end cap element. The dual-lumen over-sheath device comprises a flexible elongate member having a proximal portion and a distal portion, and a primary channel/lumen and a secondary side channel/lumen extending along the length of the elongate member. The primary channel/lumen extends along the length of the elongate member, and has an inner surface and an outer surface defined by the elongate member. The proximal portion of the primary channel/lumen is configured for housing an endoscope or a channel of an endoscopic device (e.g., a working channel of a colonoscope). The secondary channel/lumen is disposed alongside the outer surface of the primary channel. Preferably, the primary channel/lumen has a diameter greater than the diameter of the secondary channel/lumen. The primary channel can be a C-shaped or a U-shaped, or can be an enclosed channel. Likewise, the secondary channel can be a C-shaped or a U-shaped channel, or an enclosed channel.

In the various embodiments described herein, the flexible elongate member of the over-sheath devices are preferably made of a thermoplastic material comprising a helically braided configuration. In some embodiments, the helically braided configuration of thermoplastic material further includes a wound stainless steel filament wire. The end cap or semi-rigid end cap is preferably made of a material, including but not limited to, polycarbonate, polystyrene, polyamide, polyurethane, polyethylene, polypropylene or any combination thereof.

### BRIEF DESCRIPTION OF DRAWINGS

The accompanying drawings which are incorporated in and constitute a part of this specification, illustrate exemplary embodiments of the invention and together with the description serve to explain the principles of the invention. In the drawings, like reference characters generally refer to the same or similar parts throughout the different views. The drawings are not necessarily to scale, emphasis instead generally being placed upon illustrating the principles of the invention.

FIG. 1 is a drawing of an exemplary embodiment of the distal end of a dual-lumen over-sheath device according to the invention that includes a primary channel/lumen 12, secondary side channel 13 having a deflectable tip portion 16, and distal end an end cap 10.

FIG. 2 is an enlarged drawing of “DETAIL A” of the embodiment shown in FIG. 1.

FIG. 3 is a drawing of an end cap 10 according to the invention that includes a secondary side channel 13 having a deflectable distal tip 16.

FIG. 4A is a drawing depicting an exemplary embodiment of a dual-lumen over-sheath device 20 without an end cap element, the over-sheath device including a C-shaped or U-shaped primary channel/lumen 12 and a C-shape or U-shaped secondary side channel 13; FIG. 4B is an enlarged drawing of the circled portion of the device depicted in FIG. 4A.

FIG. 5 is a drawing depicting an exemplary embodiment of a dual-lumen over-sheath device that includes an end cap 10 and a secondary side channel 13 that extends along the outer surface of the primary lumen 12 of the over-sheath to the distal end of the end cap 10, and an ancillary device 18 (e.g., a retrieval basket or snare) being extended through the secondary side channel 13.

FIG. 6 is a drawing of the deflectable tip portion 16 of a secondary side channel 13 for use with the devices according to the invention, the deflectable tip portion 16 including a lumen 17 disposed within and extending along the length of an outer wall of the secondary side channel 16 for housing a pull wire.

FIG. 7 is a drawing of a dual-lumen over-sheath device according to the invention fitted over the distal end of a working channel of an endoscope and coupled to an actuator 21 (e.g., a handle) for deflecting the distal portion 16 of the secondary side channel 13 of the device.

FIG. 8 is a drawing an exemplary embodiment of a dual-lumen over-sheath device without end cap, the over-sheath device including a C-shaped primary lumen 12 and an enclosed secondary lumen 22 disposed along an outer wall of the primary lumen.

### DETAILED DESCRIPTION

Disclosed herein are devices and methods for resecting and removing tissue or aiding in the resection and removal of tissue from the digestive tract in human subjects.

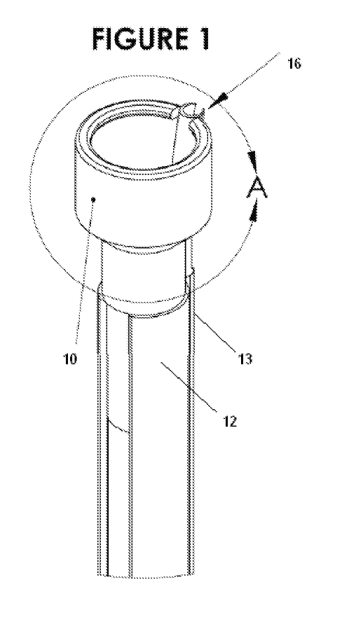

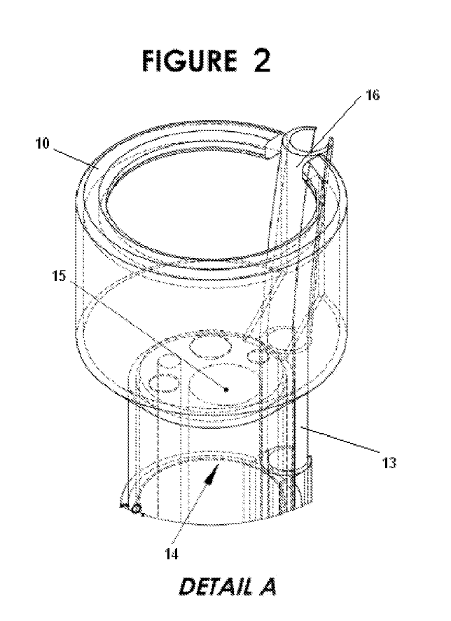

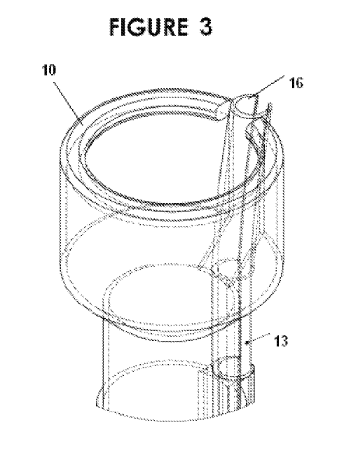

The first embodiment of the device is illustrated in FIGS. 1, 2 and 3. The device design consists of a rigid or semi-rigid end cap element 10 at the distal device end. This cap transitions to a flexible over-sheath component which contains two lumens therein. The primary lumen 12 of the over-sheath is intended to house an endoscope 14 or working channel of a colonoscope 15 while the secondary side channel/lumen 13 is intended to be used for the insertion and or retraction of ancillary devices 18 such as snares, biopsy forceps, guidewires etc.

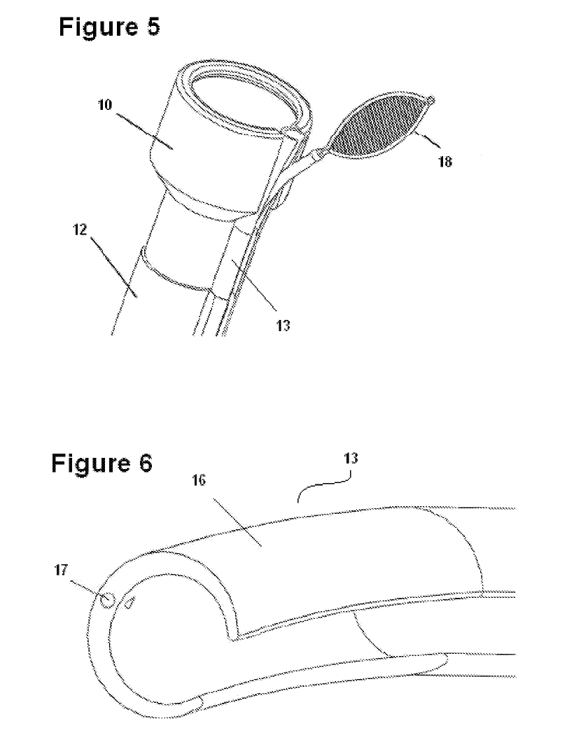

During the procedure, the physician will load the integrated end cap 10 and dual lumen over-sheath device 20 over the end of the endoscope 14 securing the end cap 10 to the end of the scope. The scope with preloaded end cap/over-sheath is then inserted to the desired anatomical site trans orally or trans rectally. Once in position the polyp or tissue sample may be elevated via media injection to elevate the mucosa and electro-cautery performed to excise the polyp. Instead of removing the entire device (end cap/over-sheath & endoscope) the physician can now pass an ancillary device 18, such as a snare or retrieval basket (e.g., Roth Basket device, US Endoscopy) device through the secondary channel 13 of the over-sheath, as shown in FIG. 5. The polyp or tissue sample is snared by the ancillary device 18 and retracted from the end of the scope. In some embodiments, the secondary channel 13 of the over-sheath is not an enclosed lumen, and has C-Channel or U-Shaped cross sectional profile. With such a profile, when the ancillary device 18 is retracted, the polyp and basket portion of the retrieval device 18 reside external to the secondary channel/lumen 13. This allows the physician to completely remove large polyps from the intended site without the necessity of having to remove the endoscope to remove the polyp sample. In this way, procedural efficiency for the removal of multiple polyps is improved.

It is preferred that the EMR cap component 10 of the device be fabricated from a clear or translucent or semi-translucent material such as, but not limited to Polycarbonate, Polystyrene, Polyamide, Polyurethane, Polyethylene, Polypropylene and/or derivatives thereof.

In the embodiment shown in FIGS. 1 through 3 inclusive, the distal end cap element 10 is not completely enclosed but has a portion of the cross section removed. This section is removed to provide for the incorporation of a deflectable tip element 16 which extends from the secondary side channel 13 to the extreme distal end of the cap element 10. This deflectable tip 16 is in constant communication with the lumen of the secondary outer channel 13 of the over-sheath and maintains a “C-Channel” or “U-Shaped” profile identical to that of the secondary over-sheath channel 13. The deflecting tip portion 16 of the embodiment may be a separate component which may be attached to the secondary channel 13 of the over-sheath or alternately may be a continuation of the secondary channel 13 of the over-sheath.

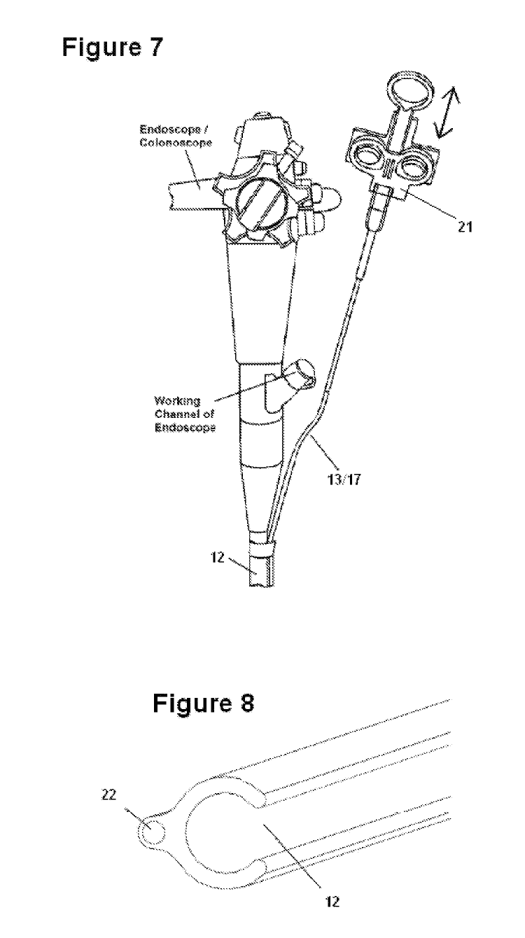

As shown in FIG. 6, the ability to deflect the distal tip portion 16 of the side channel 13 is achieved via the incorporation of a pull wire which is housed within a third lumen 17 of the over-sheath. The pull wire is anchored to the distal portion of the deflecting tip 16 via adhesive or thermal bonding techniques. The pull wire extends from the distal end of the deflecting tip portion 16 of the over-sheath in a proximal direction to an actuator 21, such as a “push-pull” handle, as illustrated in FIG. 7. Actuation of the proximal push-pull handle causes the deflecting tip 16 to deflect either outward or inward relative to the end cap 10/over-sheath device. This embodiment of the present invention may be fabricated such that the deflecting tip 16 is preferentially loaded to deflect either inward or outward at neutral. This may be controlled when the device is manufactured by lengthening the pull wire (this will result in the deflecting tip deflecting inward when at neutral) or shortening the pull wire (this will result in the deflecting tip deflecting outward when at neutral).

The ability to deflect the distal tip 16 of the secondary channel 13 is an important characteristic of the present invention as it provides the user with the ability to lift mucosa during resection to improve the quality and quantity of the sample being resected, which is often cancerous. During resection, a biopsy forceps may be passed through the outer secondary channel 13 of the over-sheath and used as described above, using the deflectable functionality of the side channel 13 to lift the mucosa.

The elongate body of the over-sheath is preferably manufactured from a thermoplastic polymer such as, but not limited to Polyurethane, Polyamide and derivatives thereof, Ether block amide copolymers, Polyimide, Polyethylene and derivatives thereof, Polytetrafluoroethylene and/or derivatives thereof. In certain embodiments, the outer shaft of the elongate over-sheath over-sheath body comprises a helically braided configuration of outer thermoplastic material such as those mentioned above with a lubricious inner liner or core, which encases the helically wound braid detail. The helically braided element may further included braided stainless steel round or rectangular filament wire wound in a “2 over 2” fashion as is known to persons skilled in the art. The incorporation of this helically wound design detail allows the user to transmit a torsional load to the over-sheath assembly from outside the patient to aid in positioning the distal end cap 10 of the device for optimal use.

An alternate embodiment of the over-sheath aspect of the present invention is illustrated in FIG. 8. In this instance, the secondary sided channel 13 of the over-sheath is of closed lumen 22 construction to provide the user with the ability to advance ancillary devices to the site of resection. In this instance, the embodiment does not incorporate a distal cap element.

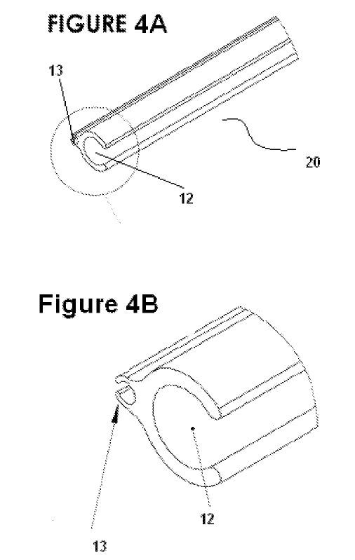

In a number of instances, the physician may have positioned the endoscope/colonoscope during routine diagnostic procedures and decide that he/she wishes to perform a resection. In this instance, this over-sheath embodiment provides the user with the ability to load the over-sheath proximally over the elongate body of the endoscope external to the patient and, using the elongate body of the endoscope, track the over sheath to the distal end of the endoscope without the need to remove the scope. In this way, the procedure may be performed more efficiently while maintaining anatomical position. The secondary channel 13 of this over-sheath embodiment may also be of “C-Channel” or “U-Shaped” configuration as shown also in FIG. 4.

It will be understood that various modifications may be made to the embodiments disclosed herein. Therefore, the above description should not be construed as limiting, but merely as illustrative of some embodiments according to the invention.

What is claimed is:

1. A device for endoscopic mucosal resection comprising,
a flexible elongate member comprising a primary channel extending along the length of the elongate member, the elongate member having a proximal portion and a distal portion, the proximal portion configured for housing a working channel of an endoscope, the distal portion comprising a rigid or semi-rigid end cap.

2. The device of claim 1, wherein the primary channel comprises a C-shape or a U-shape.

3. The device of claim 1, wherein the flexible elongate member further comprises a secondary channel disposed along an outer wall of said primary channel and extending to a distal end of said end cap, the secondary channel having a diameter smaller than a diameter of the primary lumen.

4. The device of claim 3, wherein the secondary channel comprises a C-shape or a U-shape.

5. The device of claim 3, further comprising a lumen disposed within a wall of said secondary channel, said third channel comprising a pull wire, the pull wire being coupled to a distal tip of said secondary channel and extending in a proximal direction to an actuator for deflecting the distal tip of the secondary channel.

6. The device of claim 5, wherein said actuator is a handle configured for deflecting the distal tip in an inward and an outward direction relative to the end cap.

7. The device of claim 6, wherein said distal tip is deflected in an outward direction relative to said end cap when the handle is pulled in an upward direction, and deflected in an inward direction relative to said end cap when the handle is pushed in a downward direction.

8. The device of claim 1, wherein at least a portion of an outer surface of said flexible elongate member comprises a thermoplastic material comprising a helically braided configuration.

9. The device of claim 8, wherein said helically braided configuration further comprises a helically wound stainless steel filament wire.

10. The device of claim 1, wherein said rigid or semi-rigid end cap comprises a material selected from the group consisting of polycarbonate, polystyrene, polyamide, polyurethane, polyethylene, polypropylene and a combination thereof.

11. A device for endoscopic mucosal resection comprising,
a rigid or semi-rigid end cap, said end cap configured for housing a channel of an endoscopic device, said end cap comprising a side channel disposed within or along an outer surface of said end cap.

12. The device of claim 11, wherein said side channel comprises a C-shape or a U-shape.

13. The device of claim 11, wherein said rigid or semi-rigid end cap comprises a material selected from the group consisting of polycarbonate, polystyrene, polyamide, polyurethane, polyethylene, polypropylene and a combination thereof.

14. A device for endoscopic mucosal resection comprising,
a flexible elongate member having a proximal portion and a distal portion, the elongate member comprising a primary channel and a secondary channel extending along the length of the elongate member, the proximal portion of said primary channel configured for housing a channel of an endoscopic device, said primary channel having a diameter greater than a diameter of the secondary channel, said secondary channel being disposed along an outer surface of said primary channel.

15. The device of claim 14, wherein said primary channel comprises a C-shape or a U-shape.

16. The device of claim 14, wherein said secondary channel comprises an enclosed lumen.

17. The device of claim 14, wherein said secondary channel comprises a C-shape or a U-shape.

18. The device of claim 14, wherein at least a portion of an outer surface of said flexible elongate member comprises a thermoplastic material comprising a helically braided configuration.

19. The device of claim 18, wherein said helically braided configuration further comprises a helically wound stainless steel filament wire.
